(19) United States
(12) Patent Application Publication
(10) Pub. No.: US 2014/0105976 A1
(43) Pub. Date: Apr. 17, 2014

(54) PHARMACEUTICAL FORMULATIONS OF PILOCARPINE

(71) Applicant: THERAVIDA, INC., San Mateo, CA (US)

(72) Inventors: Mehdi PABORJI, San Mateo, CA (US); Robert V. TUOHY, Norristown, PA (US); Stephen P. LEVINE, Norristown, PA (US); Dana E. MOSESON, Norristown, PA (US); Nicolas D. KIRKLAND, Norristown, PA (US)

(73) Assignee: THERAVIDA, INC., San Mateo, CA (US)

(21) Appl. No.: 14/052,302

(22) Filed: Oct. 11, 2013

Related U.S. Application Data
(60) Provisional application No. 61/712,784, filed on Oct. 11, 2012.

(51) Int. Cl. A61K 9/20; A61K 31/4178

(52) U.S. Cl. CPC A61K 9/2086; A61K 31/4178
USPC 424/465; 424/464; 514/397

(57) ABSTRACT

Disclosed herein are pharmaceutical compositions comprising at least one minitablet, where the minitablet comprises a core, comprising pilocarpine, or a pharmaceutically acceptable salt thereof; and a coating layer comprising a coating polymer.

### RELATED APPLICATIONS

The present application claims priority to the U.S. Provisional Application Ser. No. 61/712,784, filed on Oct. 10, 2012, by Paborji et al., and entitled “PHARMACEUTICAL FORMULATIONS OF PILOCARPINE,” the entire disclosure of which is incorporated by reference herein, including any drawings.

### FIELD OF THE INVENTION

The present invention is in the field of pharmaceutical formulations, and in particular formulations comprising pilocarpine.

### BACKGROUND OF THE DISCLOSURE

Pilocarpine has been used to increase salivation in patients who suffer from dry mouth in a variety of different disorders. The available formulations of pilocarpine are in immediate release form. These formulations are not suitable if a delay in the release of pilocarpine is desired.

### SUMMARY OF THE INVENTION

Disclosed herein are pharmaceutical compositions comprising at least one minitablet, where the minitablet comprises a core, comprising pilocarpine, or a pharmaceutically acceptable salt thereof; and a coating layer comprising a coating polymer.

### DETAILED DESCRIPTION OF THE EMBODIMENTS

Aspects of the present disclosure include pharmaceutical formulations comprising pilocarpine, a muscarinic agonist. The muscarinic agonist of the pharmaceutical formulations is present in a delayed immediate release formulation. Once ingested, the muscarinic agonist is not released for some time. But once the muscarinic agonist begins to be released, it is released immediately.

In the context of the present disclosure, “immediate release” or “released immediately” means that at least about 70% of the ingested active pharmaceutical ingredient in the dosage form is released from the pharmaceutical formulation within about 30-60 minutes of the ingestion of the dosage form. By “not released” or “delayed released” it is meant that less than 20% of the ingested active pharmaceutical ingredient in the dosage form is released from the pharmaceutical formulation by the time the delay is concluded and the release becomes immediate.

Throughout the present disclosure the term “about” a certain value means that a range of value±10%, and preferably a range of value±5%, is contemplated. Thus, for example, having about 70% of the active pharmaceutical ingredient (API) includes API being present between 63% and 87%, and preferably between 66.5% and 73.5%; or by way of another example, “about 45 minutes” means that the contemplated value is between 40.5 minutes and 49.5 minutes, and preferably between 42.75 minutes and 47.25 minutes.

### Pilocarpine Minitablets

Thus, in one aspect, disclosed herein are pharmaceutical compositions comprising at least one minitablet, where the minitablet comprises:

a core, comprising pilocarpine, or a pharmaceutically acceptable salt thereof; and

a coating layer comprising a coating polymer.

In some embodiments, pilocarpine is present as the free base. In other embodiments, pilocarpine is present as a pharmaceutically acceptable salt. The term “pharmaceutically acceptable salt” refers to a formulation of a compound that does not abrogate the biological activity and properties of the compound. Pharmaceutical salts can be obtained by reacting a compound of the invention with inorganic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical salts can be obtained by reacting a compound of the invention with inorganic acids such as tartric acid, oxolic acid, “carbonic acid” to form the bicarbonate or carbonate salt of the compound, acetic acid, formic acid, benzoic acid, and the like. Pharmaceutical salts can also be obtained by reacting a compound of the invention with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like. In some embodiments, the pilocarpine is pilocarpine HCl or pilocarpine nitrate.

In some embodiments, the core comprises between about 70% to about 99% of the total weight of the finally-formulated minitablet. In some embodiments, the core comprises between about 75% to about 97% of the total weight of the finally-formulated minitablet. In some embodiments, the core comprises between about 80% to about 95% of the total weight of the finally-formulated minitablet. In some embodiments, the core comprises between about 85% to about 95% of the total weight of the finally-formulated minitablet. In some embodiments, the core comprises between about 88% to about 95% of the total weight of the finally-formulated minitablet.

In some embodiments, a stock solution comprising pilocarpine, a free base thereof or a pharmaceutically acceptable salt thereof, and a polymer is prepared and then sprayed onto a fluidized bed, using methodology well-known in the art. In some embodiments, the fluidized bed is a cellulose bed. In some of these embodiments, the fluidized bed is a microcrystalline cellulose bed. In further embodiments, the fluidized bed is a silicified microcrystalline cellulose bed. In some embodiments, the fluidized bed is, for example, PROSOLV® SMCC, such as PROSOLV® SMCC 50.

In certain embodiments the core comprises further ingredients. In some embodiments, the core further comprises an osmotic agent. The osmotic agent causes the core to disintegrate rapidly and release the API as soon as the core comes into contact with an aqueous medium, such as the gastric or intestinal juice. In some embodiments, the osmotic agent is an inorganic salt. In some of these embodiments, the salt is a salt of an alkali metal. In further embodiments, the salt is a halide slat of an alkali metal. In some embodiments, the salt is selected from the group consisting of lithium chloride, lithium bromide, lithium iodide, sodium chloride, sodium bromide, sodium iodide, potassium chloride, potassium bromide, and potassium iodide.

In some embodiments, the core comprises a disintegrant. In some embodiments, the disintegrant is a crosslinked polymer. In some of these embodiments, the crosslinked polymer is crosslinked polyvinylpyrrolidone (crospovidone) or crosslinked sodium carboxymethyl cellulose (croscarmellose sodium). In other embodiments, the disintegrant is a modified starch, for example sodium starch glycolate.

In some embodiments, the core further comprises a lubricant. In some embodiments, the lubricant is a mineral, such as talc or silica. In other embodiments, the lubricant is a fat, e.g., vegetable stearin, magnesium stearate, stearic acid, or a derivatized stearic acid. In some embodiments, the derivatized stearic acid is sodium stearyl fumarate.

In some embodiments, the pilocarpine, or a pharmaceutically acceptable salt thereof, (i.e., the active pharmaceutical ingredient, or the API) is present in the core between about 0.1% to about 5% by weight of the core. In other embodiments, the API is present in the core between about 0.3% to about 4% by weight; or between about 0.5% to about 3% by weight.

In some embodiments, the fluidized bed, i.e., cellulose, in the core is present in between about 40% to about 75% by weight of the core, or between about 45% to about 70% by weight, or between about 48% to about 65% by weight of the core.

In some embodiments, the core polymer is present in between about 4% to about 15% by weight of the core, or between about 5% to about 12% by weight, or between about 5% to about 10% by weight of the core.

In some embodiments, the disintegrant is present in between about 5% to about 35% by weight of the core, or between about 5% to about 25% by weight, or between about 10% to about 30% by weight, or between about 10% to about 20% by weight, or between about 12% to about 17% by weight of the core.

In some embodiments, the salt is present in between about 10% to about 50% by weight of the core, or between about 10% to about 40% by weight, or between about 12% to about 37% by weight, or between about 15% to about 35% by weight of the core.

In some embodiments, the lubricant is present in between about 0.2% to about 2% by weight of the core, or between about 0.5% to about 1.7% by weight, or between about 0.5% to about 1.5% by weight of the core.

The core is coated by a coating layer. The coating layer delays the exposure of the core to aqueous media, for example gastric juice or intestinal fluid. The coating layer comprises a coating polymer. In certain embodiments, the coating polymer is a cellulose polymer. In some of these embodiments, the cellulose polymer is microcrystalline cellulose. In other embodiments, the coating polymer is a derivatized cellulose, for example, alkylated cellulose. In some of these embodiments, the derivatized cellulose is selected from the group consisting of ethyl cellulose, propyl cellulose and hydroxylpropyl cellulose.

In some embodiments, the application of the coating layer causes a weight gain of between about 1% to about 50% of the weight of the minitablet prior to the application of the coating layer. Thus, for example, if the weight of the core prior to the application of the coating layer is X, then after the application of the coating layer, the weight of the minitablet is 1.01X, if the weight gain is 1%, or the weight of the minitablet is 1.5X, if the weight gain is 50%. In some embodiments, the weight gain is between about 5% to about 45%. In some embodiments, the weight gain is between about 5% to about 40%. In some embodiments, the weight gain is between about 5% to about 35%. In some embodiments, the weight gain is between about 5% to about 30%. In some embodiments, the weight gain is between about 10% to about 25%.

In some embodiments, the coating polymer comprises a sugar or a polysaccharide. In some of these embodiments, the sugar or polysaccharide is selected from the group consisting of cellulose, hydroxyethylcellulose, hydroxypropylcellulose, hydroxypropylmethylcellulose, carboxymethylcellulose, maltodextrin, sucrose, modified starch, a salt of alginic acid, soluble gums, and carageenan. In other embodiments, the coating polymer comprises polyvinylpyrrolidone (PVP) or polyvinylpolypyrrolidone (PVPP).

In some embodiments, the coating polymer is a mixture of two or more polymers. In some embodiments, the mixture comprises ethylcellulose (EC) and hydroxypropylcellulose (HPC).

In some embodiments, EC is present in between about 60% to about 95% of the weight of the coating, or between about 60% to about 85% of the weight, or between about 61% to about 84% by weight, or between about 61% to about 82% by weight.

In some embodiments, HPC is present in between about 5% to about 35% of the weight of the coating, or between about 5% to about 20% of the weight, or between about 7% to about 17% by weight, or between about 7% to about 16% by weight.

In some embodiments, the coating further comprises a lubricant. In some embodiments, the lubricant is a mineral, such as talc or silica. In some embodiments, the lubricant is present in between about 1% to about 20% of the weight of the coating, or between about 5% to about 17% by weight, or between about 10% to about 16% by weight.

In some embodiments, the coating further comprises a plasticizer. In some embodiments, the plasticizer is selected from the group consisting of a phthalate-based plasticizer, a trimellitate, an adipate-based plasticizer, a sebacate-based plasticizer, an organophosphate, a maleate, a sulfonamide, a glycols or polyether, an acetylated monoglyceride, and an alkyl citrate. In some embodiments, the sebacate-based plasticiser is dibutyl sebacate (DBS). In some embodiments, the plasticizer is present in between about 1% to about 20% of the weight of the coating, or between about 5% to about 15% by weight, or between about 7% to about 10% by weight.

### Pharmaceutical Formulations

In another aspect, disclosed herein are pharmaceutical formulations comprising a sufficient number of minitablets to provide a single administrable dose to a subject. In some embodiments, a single administrable dose for pilocarpine, or a pharmaceutically acceptable salt thereof, is between 0.5-50 mg. In certain embodiments, a single administrable dose of pilocarpine, or a pharmaceutically acceptable salt thereof, is selected from the group consisting of 3 mg, 4 mg, 5 mg, 6 mg, 10 mg, 11 mg, and 12 mg. In certain embodiments, a single administrable dose is selected from the group consisting of 0.05 mg, 0.1 mg, 0.2 mg, 0.4 mg, 0.5 mg, 1 mg, 2 mg, 3 mg, 4 mg, 5 mg, 6 mg, 7 mg, 7.5 mg, 8 mg, 10 mg, 12 mg, 15 mg, 30 mg, and 60 mg. It is understood that in each single administrable dose there exists multiple minitablets. Thus, the amount of pilocarpine present in a single minitablet is smaller than that in a single administrable dose. The cumulative amount of pilocarpine in the multiple minitablets of a single administrable dose is the amount of pilocarpine in a single administrable dose.

In some embodiments, the pharmaceutical formulations are in the form of capsules. The capsules may include push-fit capsules made of gelatin, push-fit capsules, for example those made of hydroxypropylmethylcellulose, banded push-fit capsules, as well as soft, sealed capsules made of gelatin and a plasticizer, such as glycerol or sorbitol.

In some embodiments, the pharmaceutical formulations are in the form of dose sipping straws. In some embodiments, the beads are filled into a straw and a patient then drinks liquid through the straw, and through the process of drinking, the liquid pulled through the straw brings the beads into the mouth along with the liquid.

In some embodiments, the pharmaceutical formulations are in the form of dry sachets. In some embodiments, the beads are sprinkled onto food or mixed into a drink from dry sachet, and taken orally. For the dosage to be effective, the disclosed minitablets are filled into a sachet pouch, along with additional excipients needed to form a readily dispersible suspension. When the pouch is opened and the contents are poured over food or into a drink, the beads and additional excipients are mixed with the food or the drink, and form a palatable dispersion that is ingested by the subject. Excipients, such as salivants and glidants, are added for the contents to be easily swallowed with a minimum of chewing so that the coatings are not broken in the mouth.

In some embodiments, the pharmaceutical formulations are in the form of ready-to-use sachets. In some embodiments, the minitablets are premixed with an edible, high viscosity food substance (for example, yogurt, or energy gel), and the entire contents of the package is taken orally. Excipients, such as salivants and glidants, are added for the contents to be easily swallowed with a minimum of chewing so that the coatings are not broken in the mouth.

### EXAMPLES

### Example 1

### Dissolution Rate Determination

This method describes the procedure for the determination of the dissolution rate of the pilocarpine HCl formulations by using a reverse-phase, gradient, high-pressure liquid chromatography (HPLC) method, using techniques well-known in the art.

Stock solutions of pilocarpine HCl were prepared as working standards. Minitablets containing pilocarpine HCl were mixed with a fixed volume of 0.1 N HCl. At fixed time points after the mixing began, aliquots of the dissolution mixtures were injected into HPLC followed by several aliquots of the working standards. The amounts of released (dissolved) pilocarpine entities of formulations were calculated using the corresponding peak areas of pilocarpine.

A USP 2 Paddles method with the following conditions was employed to determine dissolution of various formulations.
Dissolution media: 0.1 N HCl
Agitation Rate: 50 RPM
Vessel Temp: 37° C.±0.5° C.
Sample Volume: 1.0 mL
Disso Volume: 500 mL

### Example 2

### Dissolution Rate Profiles

Prototype minitablets based on the above description were made using conventional techniques and their dissolution rates were measured. The minitablets were prepared as a series. For each series, the minitablets are designed to deliver a specified dose, expressed in terms of mg/capsule, and where the minitablets have a specific coating composition. Then a number of different sets of minitablets having different amount of coating, expressed in terms of % weight gain, were prepared and tested.

The tables below provide the data for the various tests. “% wg” refers to the amount of coating, expressed in terms of % weight gain

Sample R1

| Target dose (mg/capsule) | 3 mg |
|---|---|
| Coating (EC:HPC) | 8:2 |

Formulation

| | |
|---|---|
| Pilocarpine HCl | 10.4% |
| Prosolv SMCC 90 (filler) | 89.1% |
| Pruv (lubricant) | 0.5% |

Dissolution Profile

| | % Dissolved | % Dissolved | % Dissolved |
|---|---|---|---|
| Time point | 10% wg | 20% wg | 30% wg |
| (hour) | (n = 2) | (n = 2) | (n = 2) |
| 0 | 0.0 | 0.0 | 0 |
| 0.5 | 13.2 | 0.0 | 0 |
| 1 | 23.9 | 0.0 | 0 |
| 1.5 | 41.3 | 2.0 | 0 |
| 2 | 51.5 | 3.9 | 0 |
| 2.5 | 60.9 | 5.5 | 0 |
| 3 | 71.9 | 7.4 | 0 |
| 3.5 | 78.6 | 8.3 | 0 |
| 4 | 83.9 | 8.8 | 0 |
| 5 | 89.9 | 9.2 | 0 |
| 6 | 93.8 | 9.9 | 0 |
| 8 | 97.4 | 12.2 | 0 |
| 12 | 104.8 | 17.4 | 0 |

Sample R2

| Target dose (mg/capsule) | 3 mg |
|---|---|
| Coating (EC:HPC) | 9:1 |

Formulation

| | |
|---|---|
| Pilocarpine HCl | 10.4% |
| Ac-Di-Sol (superdisintegrant) | 10.0% |
| Prosolv SMCC 90 (filler) | 79.1% |
| Pruv (lubricant) | 0.5% |

Dissolution Profile

| | % Dissolved | % Dissolved | % Dissolved |
|---|---|---|---|
| Time point | 10% wg | 20% wg | 30% wg |
| (hour) | (n = 2) | (n = 2) | (n = 2) |
| 0 | 0.0 | 0.0 | 0.0 |
| 1 | 51.5 | 4.9 | 1.9 |
| 2 | 88.7 | 6.4 | 2.2 |
| 2.5 | 110.9 | 8.5 | 2.2 |
| 3 | 123.1 | 9.3 | 2.4 |
| 3.5 | 126.0 | 10.4 | 2.7 |
| 4 | 127.1 | 13.1 | 2.6 |
| 5 | 127.4 | 20.6 | 2.6 |
| 6 | 127.5 | 29.5 | 2.3 |
| 8 | 127.3 | 33.3 | 6.7 |

Sample R3

| Target dose (mg/capsule) | 3 mg |
|---|---|
| Coating (EC:HPC) | 9:1 |

Formulation

| | |
|---|---|
| Pilocarpine HCl | 12.5% |
| Ac-Di-Sol (superdisintegrant) | 10.0% |
| Mannitol (osmotic agent) | 50.0% |
| Prosolv | |
| SMCC 90 (filler) | 27.0% |
| Pruv (lubricant) | 0.5% |

Dissolution Profile

| | % Dissolved | % Dissolved | % Dissolved |
|---|---|---|---|
| Time point | 10% wg | 20% wg | 30% wg |
| (hour) | (n = 2) | (n = 2) | (n = 2) |
| 0 | 0.0 | 0.0 | 0.0 |
| 1 | 32.8 | 0.0 | 0.0 |
| 2 | 63.5 | 1.0 | 0.0 |
| 2.5 | 83.0 | 1.4 | 0.0 |
| 3 | 95.7 | 2.0 | 0.0 |
| 3.5 | 100.4 | 2.4 | 1.4 |
| 4 | 103.5 | 4.5 | 1.8 |
| 5 | 106.5 | 9.5 | 3.9 |
| 6 | 107.9 | 17.1 | 5.0 |
| 8 | 107.8 | 48.5 | 6.8 |

Sample R5

| Target dose (mg/capsule) | 3 mg |
|---|---|
| Coating (EC:HPC) | 8:2 |

Formulation

| | |
|---|---|
| Pilocarpine HCl | 10.4% |
| Ac-Di-Sol (superdisintegrant) | 15.0% |
| Sodium chloride (osmotic agent) | 15.0% |
| Klucel EF (HPC, binder) | 5.0% |
| Prosolv SMCC 90 (filler) | 54.1% |
| Pruv (lubricant) | 0.5% |

Dissolution Profile

| | % | % | % | % | % |
|---|---|---|---|---|---|
| | Dissolved | Dissolved | Dissolved | Dissolved | Dissolved |
| Time point | 10% wg | 15% wg | 20% wg | 20% wg | 25% wg |
| (hour) | (n = 2) | (n = 2) | (n = 2) | (n = 3) | (n = 2) |
| 0 | 0.0 | 0.0 | 0.0 | 0.0 | 0.0 |
| 1 | 30.0 | 3.0 | 4.7 | 0.4 | 0.0 |
| 2 | 86.2 | 42.3 | 19.3 | 3.5 | 1.3 |
| 2.5 | 89.8 | 73.1 | 25.7 | 13.2 | 5.7 |
| 3 | 91.3 | 86.2 | 52.1 | 42.0 | 11.9 |
| 3.5 | 92.4 | 88.7 | 79.8 | 77.2 | 37.2 |
| 4 | 93.1 | 90.3 | 89.4 | 98.3 | 72.9 |
| 6 | 94.6 | 93.5 | 93.2 | 103.5 | 103.9 |
| 8 | 95.1 | 94.0 | 94.4 | 104.1 | 105.9 |

Sample R6

| Target dose (mg/capsule) | 3 mg |
|---|---|
| Coating (EC:HPC) | 8:2 |

Formulation

| | |
|---|---|
| Pilocarpine HCl | 10.4% |
| Polyox N80 (swelling agent) | 30.0% |
| Aerosil R972 (glidant) | 2.0% |
| Klucel EF (HPC, binder) | 5.0% |
| Prosolv SMCC 90 (filler) | 52.1% |
| Pruv (lubricant) | 0.5% |

Dissolution Profile

| | % Dissolved | % Dissolved | % Dissolved |
|---|---|---|---|
| Time point | 10% wg | 15% wg | 20% wg |
| (hour) | (n = 2) | (n = 2) | (n = 2) |
| 0 | 0.0 | 0.0 | 0.0 |
| 1 | 0.4 | 1.9 | 0.5 |
| 2 | 1.7 | 1.5 | 2.4 |
| 2.5 | 2.6 | 2.1 | 1.6 |
| 3 | 3.9 | 2.8 | 2.0 |
| 3.5 | 5.5 | 3.5 | 2.4 |
| 4 | 7.4 | 4.3 | 2.9 |
| 6 | 17.9 | 8.5 | 5.1 |
| 8 | 32.5 | 14.9 | 8.6 |

Sample R8

| Target dose (mg/capsule) | 5 mg |
|---|---|
| Coating (EC:HPC) | 9:1 |

Formulation

| | |
|---|---|
| Pilocarpine HCl | 2.8% |
| Plasdone (PVP, binder) | 5.0% |
| Sodium chloride (osmotic agent) | 15.0% |
| Ac-Di-Sol (superdisintegrant) | 15.0% |
| Prosolv SMCC 90 (filler) | 61.0% |
| Pruv (lubricant) | 1.2% |

Dissolution Profile

| | % Dissolved | % Dissolved | % Dissolved |
|---|---|---|---|
| Time point | 20% wg | 25% wg | 30% wg |
| (hour) | (n = 2) | (n = 2) | (n = 2) |
| 0 | 0.0 | 0.0 | 0.0 |
| 0.5 | 0.8 | 0.0 | 0.0 |
| 1 | 3.9 | 4.0 | 2.1 |
| 1.5 | 4.9 | 9.7 | 4.2 |
| 2 | 8.0 | 11.8 | 7.1 |
| 2.5 | 9.4 | 12.4 | 7.5 |
| 3 | 15.6 | 13.4 | 7.7 |
| 3.5 | 25.0 | 16.2 | 8.5 |
| 4 | 28.2 | 17.0 | 9.2 |
| 5 | 33.2 | 17.3 | 12.9 |
| 6 | 38.5 | 17.3 | 15.5 |
| 8 | 83.9 | 25.6 | 20.0 |

Sample R9

| Target dose (mg/capsule) | 5 mg |
|---|---|
| Coating (EC:HPC) | 9:1 |

Formulation

| | |
|---|---|
| Pilocarpine HCl | 2.5% |
| Klucel EF (HPC, binder) | 7.5% |
| Sodium chloride (osmotic agent) | 25.0% |
| Ac-Di-Sol (superdisintegrant) | 15.0% |
| Prosolv SMCC 90 (filler) | 48.8% |
| Pruv (lubricant) | 1.2% |

Dissolution Profile

| | % Dissolved | % Dissolved | % Dissolved |
|---|---|---|---|
| Time point | 10% wg | 15% wg | 20% wg |
| (hour) | (n = 2) | (n = 2) | (n = 2) |
| 0 | 0.0 | 0.0 | 0.0 |
| 0.5 | 0.0 | 0.0 | 0.0 |
| 1 | 1.0 | 0.0 | 0.0 |
| 1.5 | 8.4 | 0.0 | 0.0 |
| 2 | 27.3 | 0.0 | 0.0 |
| 2.5 | 59.1 | 1.8 | 0.0 |
| 3 | 81.2 | 9.9 | 0.2 |
| 3.5 | 87.2 | 21.4 | 1.6 |
| 4 | 87.8 | 30.9 | 2.1 |
| 5 | 90.1 | 55.4 | 20.0 |
| 6 | 92.1 | 71.6 | 49.0 |
| 8 | 91.9 | 79.8 | 81.2 |

Sample R10

| Target dose (mg/capsule) | 5 mg |
|---|---|
| Coating (EC:HPC) | 8:2 |

Formulation

| | |
|---|---|
| Pilocarpine HCl | 2.5% |
| Klucel EF (HPC, binder) | 7.5% |
| Sodium chloride (osmotic agent) | 25.0% |
| Ac-Di-Sol (superdisintegrant) | 15.0% |
| Prosolv SMCC 90 (filler) | 48.8% |
| Pruv (lubricant) | 1.2% |

Dissolution Profile

| | % Dissolved | % Dissolved | % Dissolved | Dissolved |
|---|---|---|---|---|
| Time point | 10% wg | 15% wg | 20% wg | 15% wg |
| (hour) | (n = 2) | (n = 2) | (n = 2) | (n = 8) |
| 0 | 0.0 | 0.0 | 0.0 | 0 |
| 0.5 | 5.6 | 0.0 | 0.0 | 0.7 |
| 1 | 17.3 | 1.1 | 0.0 | 0.2 |
| 1.5 | 52.4 | 3.3 | 0.0 | 1.3 |
| 2 | 80.2 | 8.8 | 0.0 | 8.6 |
| 2.5 | 93.2 | 28.8 | 0.3 | 29.2 |
| 3 | 94.2 | 57.1 | 8.1 | 58.5 |
| 3.5 | 94.8 | 81.2 | 20.6 | 77.3 |
| 4 | 94.9 | 88.6 | 33.2 | 84.2 |
| 5 | 94.3 | 92.5 | 63.4 | 89.5 |
| 6 | 94.3 | 94.8 | 81.5 | 93.0 |
| 8 | 93.3 | 94.6 | 89.3 | 93.3 |

Sample R11

| Target dose (mg/capsule) | 5 mg |
|---|---|
| Coating (EC:HPC) | 8:2 |

Formulation

| | |
|---|---|
| Pilocarpine HCl | 2.5% |
| Klucel EF (HPC, binder) | 10.0% |
| Sodium chloride (osmotic agent) | 35.0% |
| Ac-Di-Sol (superdisintegrant) | 15.0% |
| Prosolv SMCC 90 (filler) | 36.3% |
| Pruv (lubricant) | 1.2% |

Dissolution Profile

| | % Dissolved | % Dissolved | % Dissolved |
|---|---|---|---|
| Time point | 10% wg | 15% wg | 20% wg |
| (hour) | (n = 2) | (n = 2) | (n = 2) |
| 0 | 0.0 | 0.0 | 0.0 |
| 1 | 5.3 | 0.0 | 0.0 |
| 2 | 37.4 | 0.0 | 0.0 |
| 2.5 | 59.5 | 2.5 | 0.0 |
| 3 | 78.8 | 5.0 | 0.0 |
| 3.5 | 90.6 | 13.4 | 0.0 |
| 4 | 96.6 | 31.1 | 1.8 |
| 5 | 100.6 | 65.0 | 23.4 |
| 6 | 100.8 | 83.2 | 57.1 |
| 8 | 100.6 | 97.1 | 93.0 |

Sample R12

| Target dose (mg/capsule) | 5 mg |
|---|---|
| Coating (EC:HPC) | 8:2 |

Formulation

| | |
|---|---|
| Pilocarpine HCl | 2.5% |
| Klucel EF (HPC, binder) | 10.0% |
| Sodium chloride (osmotic agent) | 35.0% |
| Ac-Di-Sol (superdisintegrant) | 15.0% |
| Prosolv SMCC 90 (filler) | 36.3% |
| Pruv (lubricant) | 1.2% |

Dissolution Profile

| | % Dissolved | % Dissolved | % Dissolved |
|---|---|---|---|
| Time point | 10% wg | 15% wg | 20% wg |
| (hour) | (n = 2) | (n = 2) | (n = 2) |
| 0 | 0.0 | 0.0 | 0.0 |
| 1 | 3.7 | 0.1 | 0.4 |
| 2 | 58.4 | 4.9 | 0.0 |
| 2.5 | 78.6 | 20.9 | 0.0 |
| 3 | 87.1 | 46.5 | 5.3 |
| 3.5 | 94.3 | 68.1 | 21.7 |
| 4 | 96.2 | 84.3 | 46.1 |
| 5 | 96.8 | 94.0 | 79.4 |
| 6 | 96.6 | 97.6 | 90.8 |
| 8 | 96.2 | 101.7 | 95.7 |

Sample R13

| Target dose (mg/capsule) | 5 mg |
|---|---|
| Coating (EC:HPC) | 8:2 |

Formulation

| | |
|---|---|
| Pilocarpine HCl | 2.5% |
| Plasdone K-29/32 (PVP, binder) | 7.5% |
| Sodium chloride (osmotic agent) | 25.0% |
| Ac-Di-Sol (superdisintegrant) | 15.0% |
| Neusilin UFL2 (anti-caking agent) | 2.0% |
| Prosolv SMCC 90 (filler) | 46.8% |
| Pruv (lubricant) | 1.2% |

Dissolution Profile

| | % Dissolved | % Dissolved | % Dissolved |
|---|---|---|---|
| Time point | 5% wg | 10% wg | 12.5% wg |
| (hour) | (n = 2) | (n = 2) | (n = 2) |
| 0 | 0 | 0.0 | 0.0 |
| 1 | 0 | 0.0 | 10.3 |
| 2 | 100 | 92.7 | 53.3 |
| 4 | 100 | 100.4 | 96.8 |
| 8 | 100 | 99.5 | 97.2 |
| 12 | 99 | 98.4 | 96.8 |
| | % Dissolved | % Dissolved | % Dissolved |
| Time point | 15% wg | 17.5% wg | 20% wg |
| (hour) | (n = 2) | (n = 2) | (n = 2) |
| 0 | 0 | 0.0 | 0.0 |
| 1 | 0 | 2.6 | 1.5 |
| 2 | 18 | 5.4 | 2.4 |
| 4 | 99 | 87.0 | 69.4 |
| 8 | 99 | 96.2 | 93.5 |
| 12 | 98 | 96.1 | 93.8 |

What is claimed is:

1. A pharmaceutical composition comprising at least one minitablet, where the minitablet comprises:
a core, comprising pilocarpine, or a pharmaceutically acceptable salt thereof; and
a coating layer comprising a coating polymer.

2. The pharmaceutical composition of claim 1, wherein the core comprises between about 70% to about 99%, or between about 75% to about 97%, or between about 80% to about 95%, or between about 85% to about 95%, or between about 88% to about 95% of the total weight of the finally-formulated minitablet.

3. The pharmaceutical composition of claim 1, wherein the core comprises a fluidized bed selected from cellulose or microcrystalline cellulose.

4. The pharmaceutical composition of claim 1, wherein the core further comprises a component selected from the group consisting of an inorganic salt, a disintegrant, and a lubricant.

5. The pharmaceutical composition of claim 4, wherein the salt is selected from the group consisting of lithium chloride, lithium bromide, lithium iodide, sodium chloride, sodium bromide, sodium iodide, potassium chloride, potassium bromide, and potassium iodide.

6. The pharmaceutical composition of claim 4, wherein the disintegrant is modified starch or a crosslinked polymer selected from the group consisting of crosslinked polyvinylpyrrolidone (crospovidone) and crosslinked sodium carboxymethyl cellulose (croscarmellose sodium).

7. The pharmaceutical composition of claim 4, wherein the lubricant is a mineral selected from the group consisting of talc and silica, or wherein the lubricant is a fat selected from the group consisting of vegetable stearin, magnesium stearate, stearic acid, and a derivatized stearic acid.

8. The pharmaceutical composition of claim 1, wherein the pilocarpine, or a pharmaceutically acceptable salt thereof, is present in the core between about 0.1% to about 5%, or between about 0.3% to about 4%, or between about 0.5% to about 3% by weight of the core.

9. The pharmaceutical composition of claim 3, wherein the fluidized bed, is present between about 40% to about 75%, or between about 45% to about 70%, or between about 48% to about 65% by weight of the core.

10. The pharmaceutical composition of claim 4, wherein the disintegrant is present between about 5% to about 35%, or between about 5% to about 25%, or between about 10% to about 30%, or between about 10% to about 20%, or between about 12% to about 17% by weight of the core.

11. The pharmaceutical composition of claim 4, wherein the salt is present between about 10% to about 50% by, or between about 10% to about 40%, or between about 12% to about 37%, or between about 15% to about 35% by weight of the core.

12. The pharmaceutical composition of claim 4, wherein the lubricant is present between about 0.2% to about 2%, or between about 0.5% to about 1.7%, or between about 0.5% to about 1.5% by weight of the core.

13. The pharmaceutical composition of claim 1, wherein the application of the coating layer causes a weight gain of between about 1% to about 50%, or between about 5% to about 45%, or between about 5% to about 40%, or between about 5% to about 35%, or between about 5% to about 30%, or between about 10% to about 25% of the weight of the minitablet prior to the application of the coating layer.

14. The pharmaceutical composition of claim 1, wherein the coating polymer is a cellulose polymer.

15. The pharmaceutical composition of claim 1, wherein the coating polymer comprises a sugar or a polysaccharide selected from the group consisting of cellulose, ethylcellulose (EC), hydroxyethylcellulose (HEC), hydroxypropylcellulose (HPC), hydroxypropylmethylcellulose (HPMC), carboxymethylcellulose, maltodextrin, sucrose, modified starch, a salt of alginic acid, soluble gums, carageenan, and a combination thereof, or wherein the coating polymer comprises polyvinylpyrrolidone (PVP) or polyvinylpolypyrrolidone (PVPP).

16. The pharmaceutical composition of claim 1, wherein the coating further comprises a component selected from a lubricant or a plasticizer, or a combination thereof.

17. The pharmaceutical composition of claim 16, wherein the lubricant is a mineral, selected from talc or silica, and is present between about 1% to about 20%, or between about 5% to about 17%, or between about 10% to about 16% by weight of the coating.

18. The pharmaceutical composition of claim 16, wherein the plasticizer is selected from the group consisting of a phthalate-based plasticizer, a trimellitate, an adipate-based plasticizer, a sebacate-based plasticizer, an organophosphate, a maleate, a sulfonamide, a glycols or polyether, an acetylated monoglyceride, and an alkyl citrate, and is present between about 1% to about 20%, or between about 5% to about 15%, or between about 7% to about 10% by weight of the coating.

19. The pharmaceutical composition of claim 1, wherein the composition comprises a sufficient number of minitablets to provide a single administrable dose for pilocarpine, or a pharmaceutically acceptable salt thereof, of between 0.5-50 mg to a subject.

20. The pharmaceutical composition of claim 19, wherein the single administrable dose of pilocarpine, or a pharmaceutically acceptable salt thereof, is selected from the group consisting of 0.05 mg, 0.1 mg, 0.2 mg, 0.4 mg, 0.5 mg, 1 mg, 2 mg, 3 mg, 4 mg, 5 mg, 6 mg, 7 mg, 7.5 mg, 8 mg, 10 mg, 12 mg, 15 mg, 30 mg, and 60 mg.
